(19) United States
(12) Patent Application Publication
(10) Pub. No.: US 2018/0309577 A1
(43) Pub. Date: Oct. 25, 2018

(54) SYSTEMS AND METHODS FOR HASHING OBFUSCATION

(71) Applicant: Baker Group, LLP, Portland, OR (US)

(72) Inventors: Christopher Newcombe, Beaverton, OR (US); Michael Baker, Portland, OR (US)

(21) Appl. No.: 15/961,556

(22) Filed: Apr. 24, 2018

Related U.S. Application Data
(60) Provisional application No. 62/488,952, filed on Apr. 24, 2017.

(51) Int. Cl. H04L 9/32; H04L 29/06; G06F 21/45; G06F 21/60

(52) U.S. Cl. CPC H04L 9/3236; H04L 63/083; G16H 15/00; G06F 21/602; H04L 2209/16; G06F 21/45

(57) ABSTRACT

Systems and methods for improving password security are proposed. A password entered by a user of an electronic device is hashed with a hash instructions to create a digest. The digest is then obfuscated in accordance with the methods disclosed herein by rearranging the bits comprising the digest, to create an obfuscated digest. The obfuscated digest may then be used to authenticate the user. This method decreases the likelihood of hacking or malicious interference.

### CROSS REFERENCE TO RELATED APPLICATION

The present application claims priority to U.S. Provisional Application No. 62/488,952, entitled “Systems and Methods for Hashing Obfuscation”, and filed on Apr. 24, 2017. The entire contents of the above-listed application are hereby incorporated by reference for all purposes.

### FIELD

The disclosure relates to methods for increasing the security of stored credential tokens, such as passwords, using cryptographic hash functions by rearranging the bits of the message digest.

### BACKGROUND

Authentication systems for electronic devices frequently require a user to enter a username and a corresponding password to verify their identity and be permitted access to some or all of the functions of the system. Conventionally, these systems may employ databases of usernames and corresponding passwords, but the passwords may be stored as cryptographic message digests, rather than as raw passwords, for increased security. A message digest is the result of a cryptographic hash function being applied to the passwords, which generates a unique output of a fixed length, often represented in hexadecimal. An important features of hash functions is that they are non-reversible; that is, given a password and a hashing function, one may easily compute the message digest associated with that password, but given the hashing function and a message digest, it is very difficult or impossible to reconstruct the original password which corresponds to the message digest. Thus, it is typically assumed that a database comprising a library of usernames and message digests is secure since, even if a hacker or malicious entity gained access to the database, they would be unable to reconstruct the passwords corresponding to the message digests contained therein. Thus the hacker would be unable to gain access to the system in question.

However, there is a problem with this approach. Namely, many users of a system may use common or easily-guessed passwords to secure their accounts, such as “admin”, “password1”, or “1234”. Knowing this, a hacker or malicious entity may construct a library of common passwords (which could include millions or billions of passwords), and hash these common passwords according to known methods, since many hashing functions use publicly-known algorithms. The hacker may therefore generate a library of message digests of common passwords. Then, if the hacker is able to gain access to the database of the system they want to infiltrate, they need only to compare each message digest in their library of common password digests against the library of actual password digests obtained from the database. If the hacker finds a match, then the hacker can simply look up in their common password digest library which password corresponds to the matching digest, and the hacker now has a working password. The hacker has therefore gained unauthorized access to the system.

This form of attack becomes more likely to succeed as the number of acceptable credential tokens increases. Further, security breaches of this sort may be especially dangerous in systems where a single user may have access to sensitive information regarding a large number of people. Thus, there is a need for an improved security system which is less vulnerable to the type of attack described above.

### SUMMARY

To address the above issues, the present disclosure proposes systems and methods which add an extra layer of security between password hashing and the password database. Namely, the message digests generated by hashing the passwords may be obfuscated by rearranging or shuffling the bits of the digest prior to storage and comparison in the database. This may be performed by a private method, as opposed to hash functions which are typically known to the public. In this way, a hacker may be prevented from using the hacking method described above. Even if the hacker recognized that the bits of the message digests stored in the password database have been rearranged, it may still remain difficult or impossible in practice for the hacker to gain access to the system by using brute force methods, since rearranging the bits of the digests involves a combinatorial explosion in terms of the number of possible rearrangements.

The above named objectives may be achieved by a method comprising algorithmically rearranging bits in a message digest to generate an obfuscated digest. This method would first be performed when the message digest was originally stored, then performed again each time an authentication token was received.

The method may further comprise receiving a password and generating the message digest with a cryptographic hash function. The obfuscated digest may be compared to an entry in a database, a user may be authenticated in response to the obfuscated digest matching the entry, and the user may not be authenticated in response to the obfuscated digest not matching the entry. The rearranging comprises moving a first bit from a first position to a second position, the first position often different from the second position. The rearranging may include, for each bit in the message digest, moving the bit from an initial position to a final position, the initial position often being different from the final position. For example, in one instance of the rearranging, each bit in the message digest may be moved from an initial position to a different final position. In another instance of the rearranging, only a first subset of the bits (e.g., a majority of the bits) may be moved from an initial position to a different final position, while a second subset of bits end up in a respective same position (e.g., are moved back to the same initial position) after performing the rearranging. The rearranging is performed according to a defined procedure, the defined procedure not being publicly known, and/or the rearranging may be performed according to an injective function.

In another embodiment, the above-named objects may be achieved by a method, comprising assembling a medical report only in response to authenticating a user based on an obfuscated hash digest. The method may further comprise receiving a password from the user, hashing the password to generate a hash digest, and rearranging the bytes of the hash digest to generate the obfuscated hash digest. The authenticating may include comparing the obfuscated hash digest to a stored obfuscated hash digest, and the user may be authenticated only in response to the obfuscated hash digest matching the stored obfuscated hash digest. In some examples, the medical report may relate to an endoscopic procedure, and the medical report may include one or more of patient data, procedure information, and image data. When the medical report includes image data, the image data may be retrieved from a medical instrument comprising an endoscope. When the medical report includes procedure information, and the procedure information may be manually entered at a terminal.

In yet another embodiment, the above-named objects may be achieved by a system. The system may include a terminal; a database including a plurality of usernames and a plurality of obfuscated hash digests; a processor communicatively coupled to the terminal and the database; instructions stored in non-transitory memory executable by the processor to: receive a password at the terminal from a user; hash the password to generate a hash digest; rearrange the bytes in the hash digest to generate an obfuscated hash digest; compare the obfuscated hash digest to one of the plurality of obfuscated hash digests in the database; authenticate the user based on the comparison; and assemble a medical report in response to the authentication. Assembling the medical report may include retrieving image data from an endoscopic instrument, retrieving patient data from a second database, and retrieving procedure information from an input device. The instructions may be further executable by the processor to: alert a system administrator if the user is not authenticated. In some examples, the rearranging may be performed according to a private function stored locally in the non-transitory memory, and wherein the private function may be an injective function.

The processes described herein may be considered valid regardless of where each bit is moved, whether some bits remain in the respective original position, if the bits are moved in groups, how many groups of bits there are, or whether each group of bits includes the same number of bits. The processes described herein may provide fixed or variable obfuscation. For example, processes that are described herein include an algorithm for rearranging bits within the hashed digest. The algorithm states how that rearrangement is accomplished so the rearrangement can be repeated the next time the hashed token is to be obfuscated. In one implementation, a fixed obfuscation may be performed when there is a single pattern of rearrangement for all tokens. In another implementation, a variable obfuscation may be performed when the rearrangement is based on any information known about the submitted token. Examples could include, but are not limited to characters in the token and the origin of the token (e.g., where the token came from), such as the IP address for a token submitted over the Internet/an intranet. Note that in this example a user submitting from a different IP address is likely to cause a failed match with the stored, obfuscated hash digest even if the underlying token submitted was correct.

Additional details and non-limiting examples of practical applications of the systems and methods described above will become apparent from the following disclosure.

### BRIEF DESCRIPTION OF THE DRAWINGS

The disclosure may be better understood from reading the following description of non-limiting embodiments, with reference to the attached drawings, wherein below:

FIG. 1 shows an example technical environment which may employ one or more of the methods disclosed herein;

FIG. 2 shows an example data flow diagram for the medical report generation system in accordance with HL7 protocol;

FIG. 3 shows an example method for generating endoscopic reports;

FIGS. 4 and 5 schematically illustrate example hashing digest obfuscations; and

FIG. 6 shows an example method for carrying out message digest obfuscation which may be performed as part of another method.

### DETAILED DESCRIPTION

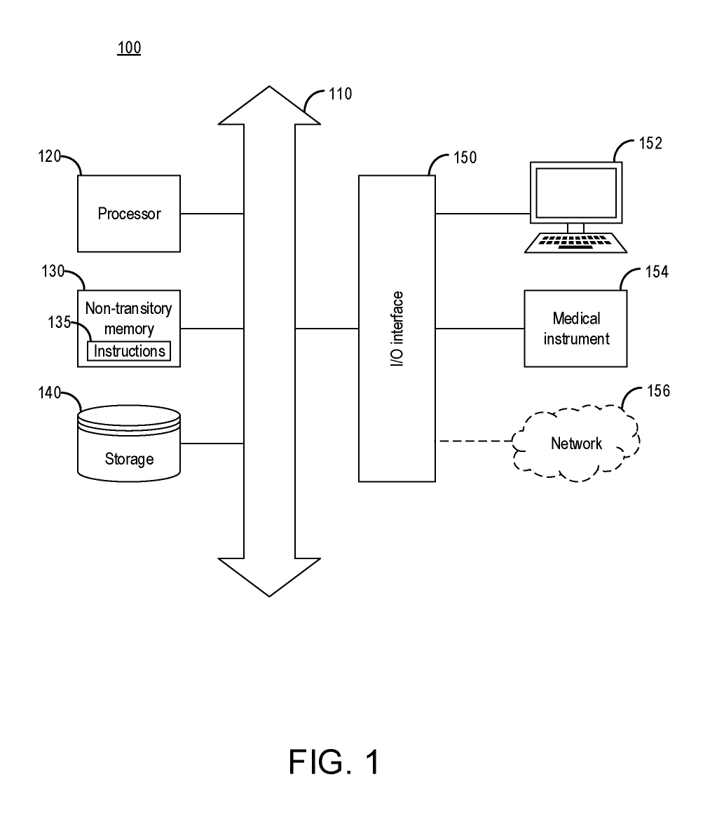

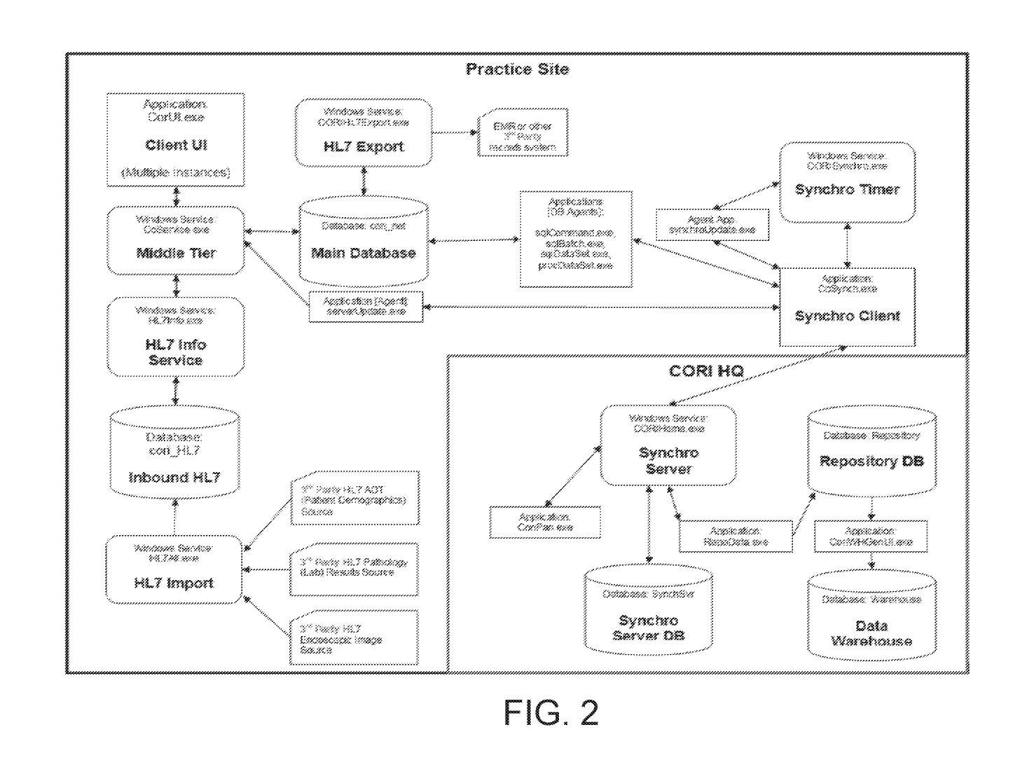

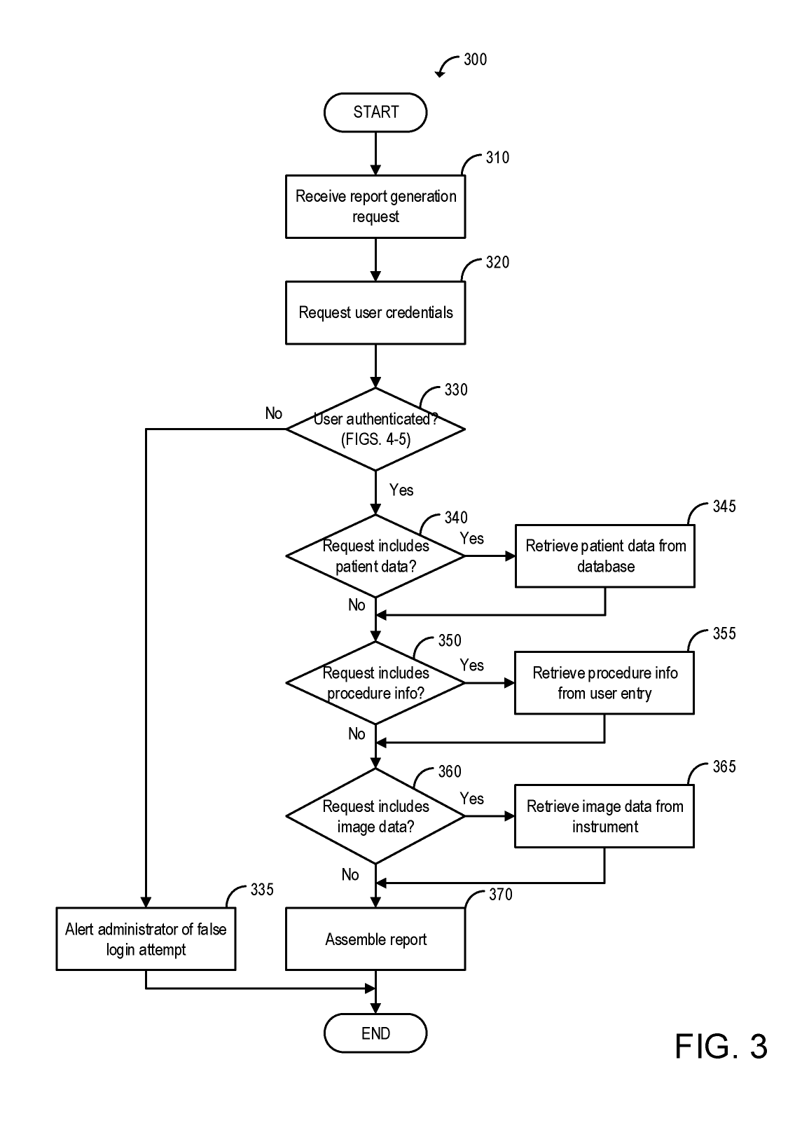

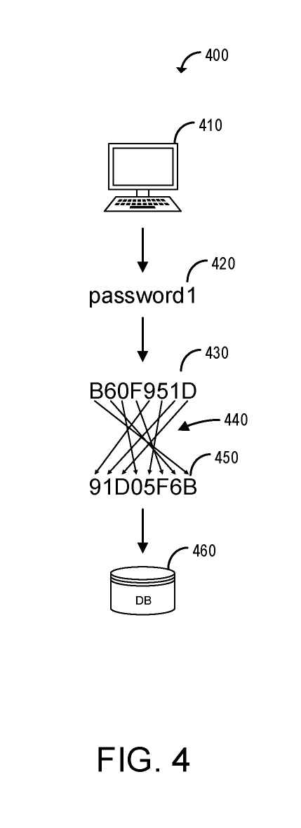

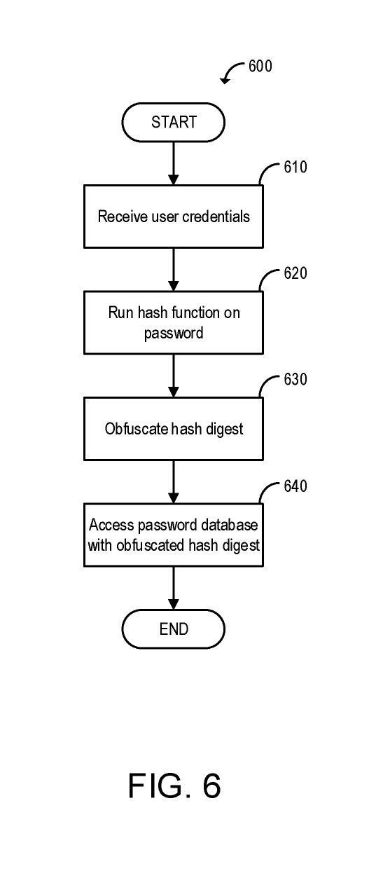

The following disclosure relates to systems and methods for increased password securing using hashing and/or message digest obfuscation. These methods may be used in any suitable computing environment, including medical reports generation systems. FIG. 1 depicts an example system for generating medical reports, such as reports summarizing endoscopic procedures, which may be employed to execute one or more of the methods described herein. FIG. 2 depicts an example of electronic medical records communication and data flow in accordance with Health Level 7 (HL7) protocol, which may be carried out by the system of FIG. 1. One example method which may be executed by said system is illustrated in FIG. 3, which describes procedures for receiving a medical report request and retrieving and assembling the data associated with said request. FIG. 4 schematically depicts password hashing and obfuscation by rearrangement of the bytes in the message. FIG. 4 schematically depicts password hashing and obfuscation by rearrangement of the bits in the message digest, which may increase password security in authentication methods, such as the method of FIG. 6. FIG. 6 depicts a method which may comprise part of the method of FIG. 3, to authenticate a user based on an obfuscated and hashed password.

FIG. 1 depicts an example medical report generation system 100 which may execute one or more of the methods described below. System 100 may include a communications bus 110 which is communicatively coupled to one or more other components. In this example, the communications bus 110 is in communication with a processor 120, non-transitory memory 130 including instructions 135, a storage device 140, and an input/output (I/O) interface 150. One or more components, such as terminal 152, medical instrument 154, and network 156, may be communicatively coupled to the I/O interface, and thus to the communications bus 110 therethrough. Communications bus 110 may enable the various components of system 100 to communicate with one another via appropriate electronic communications protocols, such as those discussed in greater depth below.

System 100 includes processor 120, which is able to receive and execute computer-readable instructions. Processor 120 may comprise a local or non-local processor. In one example, processor 120 may include a plurality of parallelized processors configured to execute instructions written and/or optimized for parallel computation. In another example, processor may include a plurality of non-local processing devices, e.g. in a non-local computing cloud. Processor 120 may receive instructions and data from a plurality of sources, including non-transitory memory 130, storage device 140, terminal 152, medical instrument 154, and optional network 156. Processor 120 may be used to execute one or more of the methods disclosed below, such as methods 300 and 600.

System 100 further includes non-transitory memory 130. Non-transitory memory 130 may comprise an appropriate form of non-volatile memory, such as HDD, SSD, CD, DVD, or other appropriate storage device. Non-transitory memory may be held locally or non-locally, and may be provided with instructions 135 which are computer-readable and -executable. Instructions 135 may include instructions representative of methods 300 and 600, for example.

System 100 further includes storage device 140. Storage device 140 may be a local or non-local storage device which stores information useful for carrying out the methods described herein. For example, storage device 140 may store patient data such as patient's name and personal information, medical history, insurance information, scheduled appointments, attending physicians, etc. Storage device 140 may also store information regarding current procedures, such as dates, times, attending physicians and staff, diagnoses, notes, etc. Further, storage device 140 may store image data related to one or more medical procedures, such as digital photographs generated by a digital camera during an endoscopic procedure. Storage device 140 may comprise a HDD, SSD, CD, DVD or other non-transitory memory device; however, in another example, the storage device may include transitory or volatile memory such as RAM or KAM without departing from the scope of this disclosure. Storage device 140 may store data in an encrypted or password-protected format, and/or it may store said data in accordance with one or more standard protocols for managing electronic medical communications, such as HL7 or DICOM. Further, in some examples, storage device 140 may include a plurality of storage devices, and/or may be embodied as cloud storage, for example.

Storage device 140 may also include a library or database of usernames and passwords for user authentication and identity verification in accordance with the methods described herein. Thus, the database may store the passwords only in a hashed and obfuscated form. That is, the passwords entered by users into system 100 may be hashed according to a hashing function to generate a hash and/or message digest, and the digits or bytes of the hash and/or message digest may be rearranged to generate an obfuscated hash and/or message digest. Only these obfuscated hash and/or message digests may be stored in storage device 140. Then, upon user request for authentication, the obfuscated hash and/or message digests stored in the storage device may be compared to an obfuscated hash and/or message digest based on a user-entered password in order to authenticate the user. These methods are discussed in greater detail below.

System 100 may include input/output (I/O) interface 150. I/O interface is a communicative interface which can communicate between communications bus 110 and one or more external components. In some examples, system 100 may employ multiple I/O interfaces, for example one interface for each external device. In this example, I/O interface 150 is communicatively coupled to terminal 152, medical instrument 154, and optionally network 156. I/O interface may receive, relay, transduce, or translate information from the external components to the communications bus 110 and vice versa. In this way, processor 120 may operate on data or instructions which are stored or retrieved non-locally or external to system 100 itself.

Terminal 152 may comprise a computer terminal communicatively coupled to system 100 and provided with instructions enabling a user to interact with system 100 from terminal 152. This may enable a user to log in, retrieve data, generate endoscopic reports, etc. A user may interact with terminal 152 via a keyboard and mouse and receive information via a screen and speakers, for example. In other embodiments, terminal 152 may comprise a laptop, smartphone or other mobile device, tablet, server, or other appropriate device. Terminal 152 may be communicatively coupled to the I/O interface 150, and thence to communications bus 110, by LAN, wireless, satellite, Bluetooth, Internet, or other appropriate method. In another example, a user may interact with system 100 directly by input and output devices, without the need to use an external client terminal.

I/O interface 150 may also be communicatively coupled to one or more medical instruments 154. In one example, medical instrument 154 may comprise an endoscope or endoscopic instrument, configured to be introduced into a patient's body. The endoscope may be provided with a camera which can generate images. I/O interface 150 may therefore be configured to retrieve images generated by medical instrument 154 and deliver them to processor 120 or storage 140, for example. Additionally or alternatively, medical instrument 154 may comprise EEG, EKG, CT, PET, fMRI, ultrasound, or other appropriate medical instruments, in which case I/O interface may be configured to receive and record brain activity, heart rate, imaging data, etc.

I/O interface 150 may optionally be connected to network 156. In one example, network 156 may comprise a local network. In another example, network 156 may include a connection to the Internet or cloud computing resources. However, connection to network 156 is not required and, for reasons of security and patient confidentiality, it may be preferred in some applications not to include network 156.

As mentioned above, medical reports generation system 100 may be compliant with one or more standards or protocols relating to electronic medical communication. FIG. 2 illustrates data flow which may be performed by system 100 during one or more of the methods disclosed herein, in accordance with one such protocol. System 100 may be compliant with Health Level 7 (HL7) protocol for the transfer of medical and administrative data between different software applications, different hardware devices, and/or different healthcare providers. As another example, system 100 may be compliant with Digital Imaging and Communications in Medicine (DICOM) standards for storing, transmitting, and handling medical imaging data, such as data generated by medical instrument 154. In particular, when medical instrument 154 comprises an endoscope, images or photographs of a patient's body may be taken to aid in diagnostics and ease of communication; these images may be communicated according to DICOM protocol. FIG. 2 therefore shows one example of a data flow according to HL7 protocol. As HL7, DICOM, and related electronic medical communications protocols will be well understood by one with skill in the art, they will not be described further herein.

FIG. 3 depicts an example method 300 of medical report generation according to the present disclosure. Method 300 may be executed by system 100, for example, in response to a user request to generate an endoscopic report. As discussed above, endoscopic reports, or other medical reports, may include data from a plurality of different sources, including image data retrieved from a medical instrument, diagnostic data entered by a physician, technician, or other user, patient data stored in a database, and so forth. Much of this data may be HIPAA protected or otherwise be sensitive data which requires strong security measures. Thus method 300 includes user authentication in accordance with the present disclosure, employing hash and/or message digest obfuscation, to improve security and to decrease the likelihood of a hacker or other malicious entity from gaining access to said data. Method 300 may therefore generate a medical report only in response to a user being authenticated on the basis of an obfuscated hash and/or message digest matching a stored obfuscated hash and/or message digest, the obfuscated hash and/or message digest being computed based on the user's password. This may substantially increase security and user confidence, as discussed in greater depth below.

Method 300 begins at 310, where a report generation request is received. The request may be issued by a user of system 100 or may be generated automatically in response to certain control actions, such as the completion of a medical procedure. The report generation request may include specified data such as patient information, images, physician notes, and other information. Once the request is received, processing proceeds to 320.

At 320, the method requests user credentials for authentication. Although in this example the user credentials are requested after the report generation request is received, in another example this order may be reversed (e.g. to prevent unauthorized users from issuing report generation requests). The user credential request may be sent to a terminal, e.g. terminal 152, where the user is interacting with the system. The credential request may include a request for a username and a password. Once the user has entered their credentials, processing proceeds to 330.

At 330, the method evaluates the user credentials to verify the user's identity. Authenticating a user may be performed using cryptographic hashing and hashing obfuscation techniques to increase security. These methods are described in greater detail below with reference to FIGS. 4-6. In response to the user being authenticated, processing proceeds to 340, whereas in response to the user not being authenticated, processing proceeds to 335.

At 335, in response to the user not being authenticated, the method may include alerting a system administrator of the failed login attempt. In some examples, the alert may only be issued after a threshold number of failed login attempts, e.g. 3 failed login attempts. The alert may include the username and location (e.g. which terminal) of the attempted login. In some examples, the alert may include the password used for the attempted login. The alert may provide options for the administrator to deactivate the account corresponding to the username, if such an account exists, or the account may be deactivated automatically until administrator review. After the alert is sent, method 300 returns.

At 340, in response to the user being authenticated, method 300 may begin retrieving data necessary for assembling the endoscopic report requested. At 340, the method checks if the request includes a request for patient data. If so, processing proceeds to 345 where patient data is retrieved. Patient data may include information regarding a patient's medical history, personal information, insurance information, etc. Patient data may be stored in, and retrieved from, a dedicated database such as storage device 140. In another example, patient data may be retrieved via network 156. Once patient data has been retrieved and/or loaded into working memory, processing may proceed to 350. Conversely, if the request does not include a request for patient data, processing may proceed directly from 340 to 350.

At 350, the method checks if the request includes a request for procedure information. If so, processing proceeds to 355 where procedure information is retrieved. Procedure information may include information regarding a current procedure being performed on the patient or a recently performed procedure. This information may specify the type of procedure, the duration, and the attending physician and other staff. This information may also include notes or observations entered by a user regarding the procedure, for example a diagnosis or an observation warranting further study. Procedure information may take the form of text, diagrams, images, voice memos, etc. This information may be entered by one or more users using input devices such as a keyboard, mouse, trackpad, touchpad, microphone, etc. The entered information may be stored locally in working memory or may be retrieved from a database, e.g. by I/O interface 150 or by network 156. Once procedure information has been retrieved, processing may proceed to 360. Conversely, if the request does not include a request for procedure information, processing may proceed directly from 350 to 360.

At 360, the method checks if the request includes a request for image data. If so, processing proceeds to 365 where image data is retrieved. Image data may include raw image data or image files which have been processed and/or compressed. In one example, image data may be stored and transmitted in a conventional file format, such as JPEG, GIF, TIFF, PNG, etc. Image data may be retrieved directly from a medical imaging device, such as medical instrument 154, in one example. However, in other embodiments, the image data may be generated by the medical instrument and stored locally or non-locally before the report generation request is made. In this case, the method may retrieve the image data e.g. from an image database. Once the image data has been retrieved and/or loaded into working memory, processing may proceed to 370. Conversely, if the request does not include a request for image data, processing may proceed directly from 360 to 370.

At 370, the method may assemble the report. This may include assembling one or more of patient data, procedure information, and image data into a report. Other data sources may also be employed in accordance with the capabilities of system 100 and the user request. The method may assemble the report into a human-readable format. The report may be saved in an appropriate format (e.g. PDF) in working memory or in a database or storage device. The report may be presented to the user on a screen of a terminal and/or may be printed or sent to an appropriate location according to user commands. Once the method has assembled the necessary data and generated the report, method 300 return.

The medical reports generation process described above has a need for a high level of security in storing user passwords. Thus, FIGS. 4-6 present example methods for obfuscating hashed passwords in order. FIGS. 4-6 present methods for obfuscating hashed passwords to add an additional layer of security beyond conventional hash functions. FIG. 4 schematically depicts a method 400 according to the present disclosure for obfuscating a message digest to increase password security. A user may interact with one or more of the systems discussed herein via terminal 410. Terminal 410 may request authentication credentials from a user before allowing the user to interact with the system, or before giving the user access to certain features of the system. For example, at terminal 410, a user may be asked for a login including a username and a password.

A user may enter a password 420 into terminal 410. In this case, an example password 420 is given as “password1”. Accordingly, the terminal 410 (or an associated message digest obfuscating system) receives a message of “password1”. This password may then be entered into a cryptographic hash functions, such as MD5, SHA-2, or other appropriate hash function to create a message digest. Hashing may be performed (e.g., applied to the message) locally on terminal 410 by one or more processors, and be stored on one or more non-volatile memories. The unhashed password may be transmitted to a server with which the terminal 410 is connected, e.g. by a secure connection, and the hash may then be computed non-locally.

In this example, the password “password1” may have a hypothetical 8-bit hashing function applied to it, which generates the message digest 430, “B60F951D”. Digest 430 is presented here in hexadecimal notation, where each digit (0-F) represents one bit of the 8-bit digest, for ease of explanation. In other examples, the digest may be represented in other bases, such as binary, octal, or decimal, without departing from the scope of this disclosure.

After hashing, message digest 430 may undergo an obfuscation process 440 in accordance with this disclosure (e.g., obfuscation may be applied to the message digest by the terminal 410 or associated message digest obfuscating system), resulting in an obfuscated digest 450. In the obfuscation process, the individual bits in digest 430 may be rearranged according to a known procedure. In one example, each bit in the digest may be in a different position in the digest 430 as compared to the obfuscated digest 450. However, in other examples, some but not all of the bits may be rearranged. Obfuscation process 440 may be performed according to a procedure which is stored in the memory of the medical report generation system, but may not be publicly known. In this way, it may be very difficult for a hacker or malicious entity to guess how the rearrangement is performed. In general, for a message digest of N bits, there are N! possible rearrangements of the digest. For example, using a SHA-512 hashing algorithm results in a 512-bit, or 64-byte, message digest which has 64!≈1.27×10⁸⁹ possible rearrangements even if limited to moving groups of bits around in standard bytes. This method results in a number of possible rearrangements too large to be addressed using simple brute force tactics.

The obfuscation process 440 may be conducted according to instructions stored on one or more non-volatile memories and executed by one or more processors. These memories and processors may be located at one or more locations. For example, the obfuscation process 440 may take place locally on a terminal with instructions stored on a terminal memory and being executed by a terminal processor. However, the obfuscation process may also take place remotely relative to the terminal. The terminal may send the password to a different machine where the obfuscation process 440 is conducted. The machine receiving the password will conduct the obfuscation process 440 according to instructions stored on one or more non-volatile memories and executed by one or more processors. Furthermore, the hashing process 430 and obfuscation process 440 may take place on a same or different machine. Many combinations of storage locations, execution locations, hashing locations and obfuscation locations are possible without departing from the scope of the disclosure.

Obfuscation process 440 may be selected at the time of manufacture or time of set-up and configuration of the systems and methods described herein. Further, each individual system may be given a unique obfuscation function, to prevent malicious users from learning the obfuscation by repeated testing of one system, and then applying that knowledge to another system. The obfuscation process may be performed based on an injective or “one-to-one” function in which each bit in the digest is mapped to one and only one bit of the obfuscated digest, and vice versa. The obfuscation function may be stored in the memory of system 100 as computer-executable instructions, a mathematical function, or as another appropriate representation. In such an embodiment, the obfuscation process 440 may take place locally stored on local memory and executed by local a local processor or processors.

Obfuscation process 440 may take place in the same location as hashing (e.g. either locally on terminal 410 or non-locally at a server). In this case, the message digest 430, “B60F951D”, has its hexadecimal digits rearranged to create obfuscated hash 450, “91D05F6B.” In this case, each hexadecimal digit of the digest 430, representative of one bit, takes on a new position in the obfuscated digest 450. After the obfuscated digest is generated, the method may proceed to authenticate the user at a database 460 for example, using the username and the obfuscated digest, in place of an ordinary (non-obfuscated) message digest. The obfuscated digest based on the password entered by the user may be compared to an obfuscated digest stored in the memory of database 460, determined to correspond to the entered username. If the obfuscated digest matches that stored in the database, the user is authenticated and allowed to use the system as normal, for generating medical reports and other functions described elsewhere herein. If the obfuscated digest does not match that stored, the user is not authenticated and may be declined access to the system. In another example, if it is a user's first time using the system, the obfuscated digest may be stored in the database 460 corresponding to the user's username for future authentication. Once the obfuscated digest is stored in the database, future tokens (e.g., passwords or messages) that are received may be hashed and obfuscated, as described above at 430 and 440, then compared to the stored obfuscated digest (e.g., obfuscated digest stored in database 460 as described above). For each future token received, if the token matches the stored version, the token is accepted (e.g., the user is allowed access to the system or is otherwise authenticated), and if the token does not match the stored version, the token is rejected (e.g., the user is not allowed access to the system or is otherwise not authenticated, and any associated alerts are issued).

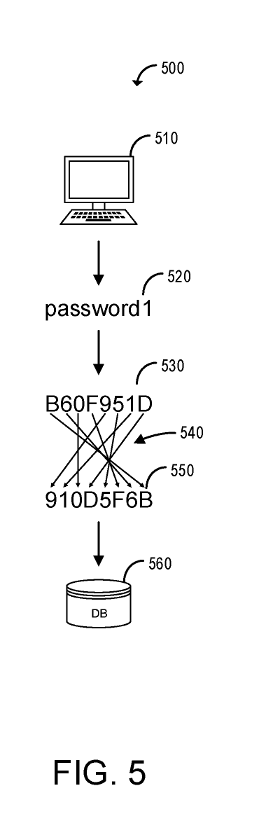

FIG. 5 schematically depicts a method 500 according to the present disclosure for obfuscating a message digest to increase password security. Method 500 may be largely the same as method 400 of FIG. 4, except that the obfuscation process may be different and/or result in an obfuscated hash/message in which at least one bit maintains the same location before and after obfuscation. Accordingly, the description of FIG. 5 will focus on the differences between method 400 and 500. For example, the description of terminal 410 may correspond to a terminal 510, the description of password 420 may correspond to a password 520, the description of message digest 430 may correspond to a message digest 530, and the description of database 460 may correspond to database 560. The message digest 530 may undergo a different obfuscation process 540 (relative to that described in FIG. 4) or the obfuscation process 540 may otherwise result in a different obfuscation of the bits of the message digest 530 than described in FIG. 4. A resulting obfuscated digest 550 (e.g., an obfuscated hash) results from the obfuscation process 540. In this case, all but one hexadecimal digit of the digest 530, each representative of one bit of the message digest, takes on a new position in the obfuscated digest 550. The third bit of the message digest (“0”) is shown as occupying the same location in both the message digest 530 and the obfuscated digest 550 in this example.

By using an additional step in the authentication process, wherein the bits of a message digest are rearranged or “scrambled” according to private function, security of the system may be increased. In particular, this method may help prevent the sorts of attacks described above in the Background section. These attacks are deterred by greatly increasing the amount of potential combinations.

These features described above may be embodied by a method, such as method 600 shown in FIG. 6. Method 600 may be executed by system 100, for example, as part of a larger user authentication process, or as part of a larger user interaction with the system. In one example, method 600 may be performed as part of method 300, e.g. at block 330. Thus, the authentication based on the obfuscated message digest may be required for the user to generate a medical report using system 100, for example. In other embodiments, said authentication methods may be required for other user interactions with system 100. These authentication methods are not, however, limited to such applications, and may be employed in other technical environments or circumstances without departing from the scope of this disclosure. Method 600 may be performed immediately upon a user attempting to interact with the system, or may only be performed when a user attempts to perform an action which requires some amount of security clearance (e.g. when accessing HIPAA protected data).

Furthermore, the processes may take place at various locations. The hashing and obfuscation processes may take place on general devices, medical devices, terminal, servers, cloud locations, or many other machines without departing from the scope of the disclosure. The process are performed according to instructions stored on one or more non-volatile memories and executed by one or more processors, both of which may also be at various locations. Many combinations of machines can be used without departing from the scope of the disclosure. Furthermore, the processes described may increase the security of a large network of machines by greatly increasing the possible combination for a password.

Method 600 begins at 610 by receiving user credentials. These credentials may be received in response to a user authentication request, whereupon the user enters credentials comprising a username and corresponding password. Upon receiving the user credentials, the method may proceed to 620.

At 620, the method processes the received password according to a cryptographic hashing function, such as those described above. The hashing function is performed according to instructions stored on one or more non-volatile memories and executed by one or more processors. The hash function produces a unique, non-reversible message digest. Processing then proceeds to 630.

At 630, the method obfuscates the message digest to produce an obfuscated digest. This may be performed by rearranging or “scrambling” the bits of the digest according to the methods described above. The obfuscation process is performed according to instructions stored on one or more non-volatile memories and executed by one or more processors. Once the obfuscated digest is produced, processing process proceeds to 640.

At 640, the method uses the username and obfuscated digest to access the database in question, e.g. a database where user credentials are stored. User credentials stored in the database may comprise obfuscated message digests corresponding to each username. The method may then compare the obfuscated digest generated by the method to that stored in the database. If the obfuscated digests match, the user is authenticated; if not, the user is not authenticated. The method then returns.

This disclosure therefore provides for systems and methods which employ message digest obfuscation as a method for improving password storage security. Thus, employing message digest obfuscation by rearranging bits in a message digest before storage in a database and/or before authentication may be used as part of a user identity verification process performed as part of an endoscopic report generation routine.

The password security is increased by greatly increasing the number of possible combinations of a password. Rearranging the bits of the password creates different possibilities for each individual bit. When applied to a hashed password, this creates a combinatorial explosion of potential passwords. This improved security may benefits large groups of machines. The obfuscation process may be employed to authenticate users on large networks of machines, such as in the medical field. Furthermore, the obfuscation process can take place on various combinations of machines which may further enhance security.

The description of embodiments has been presented for purposes of illustration and description. Suitable modifications and variations to the embodiments may be performed in light of the above description or may be acquired from practicing the methods. The methods may be performed by executing stored instructions with one or more logic devices (e.g., processors) in combination with one or more additional hardware elements, such as storage devices, memory, hardware network interfaces/antennas, switches, actuators, clock circuits, etc. The described methods and associated actions may also be performed in various orders in addition to the order described in this application, in parallel, and/or simultaneously. The described systems are exemplary in nature, and may include additional elements and/or omit elements. The subject matter of the present disclosure includes all novel and non-obvious combinations and sub-combinations of the various systems and configurations, and other features, functions, and/or properties disclosed.

As used in this application, an element or step recited in the singular and proceeded with the word “a” or “an” should be understood as not excluding plural of said elements or steps, unless such exclusion is stated. Furthermore, references to “one embodiment” or “one example” of the present disclosure are not intended to be interpreted as excluding the existence of additional embodiments that also incorporate the recited features. The terms “first,” “second,” and “third,” etc. are used merely as labels, and are not intended to impose numerical requirements or a particular positional order on their objects. The following claims particularly point out subject matter from the above disclosure that is regarded as novel and non-obvious.

1. A method, comprising rearranging bits in a message digest to generate an obfuscated digest.

2. The method of claim 1, further comprising receiving a password and generating the message digest with a cryptographic hash function.

3. The method of claim 1, further comprising comparing the obfuscated digest to an entry in a database,
authenticating a user in response to the obfuscated digest matching the entry, and
not authenticating the user in response to the obfuscated digest not matching the entry.

4. The method of claim 3, wherein the rearranging is performed according to an injective function, wherein responsive to the authenticating, protected data is presented or made available to a user.

5. The method of claim 4, further comprising receiving a password from the user, hashing the password to generate the message digest, and rearranging the bits of the message digest to generate the obfuscated message digest.

6. The method of claim 5, wherein the authenticating includes comparing the obfuscated message digest to a stored obfuscated message digest, wherein the user is authenticated only in response to the obfuscated message digest matching the stored obfuscated message digest.

7. The method of claim 1, wherein the rearranging comprises moving a first bit from a first position to a second position, the first position different from the second position.

8. The method of claim 1, wherein the rearranging comprises, for each bit in the message digest, moving the bit from an initial position to a final position, the initial position being different from the final position.

9. The method of claim 1, wherein the rearranging is performed according to a defined procedure, the defined procedure not being publicly known.

10. A system, comprising:
a terminal;
a database including a plurality of usernames and a plurality of obfuscated message digests;
a processor communicatively coupled to the terminal and the database;
instructions stored in non-transitory memory executable by the processor to:
receive a password at the terminal from a user;
hash the password to generate a message digest;
rearrange the bits in the message digest to generate an obfuscated message digest;
compare the obfuscated message digest to one of the plurality of obfuscated message digests in the database;
authenticate the user based on the comparison; and
assemble a medical report in response to the authentication.

11. The system of claim 10, wherein the rearranging is performed according to a private function stored locally in the non-transitory memory, and wherein the private function is an injective function.

12. A method, comprising:
hashing a message digest according to hashing instructions;
moving a first bit of the message digest from a first position to a second position;
moving a second bit of the message digest from a first position to a second position;
the second positions of the first bit and second bit being determined by obfuscating instructions; and
the hashing instructions and obfuscating instructions being stored on one or more non-volatile memories and executed by one or more processors.

13. The method of claim 12, including, moving a plurality of bits of the message digest from respective first positions to respective second positions and the respective second positions being determined by the obfuscating instructions.

14. The method of claim 12, including, maintaining a third bit in a first position.

15. The method of claim 13, including, maintaining a plurality of bits in respective first positions.

16. The method of claim 12, including, after the hashing and moving steps are performed, comparing the message digest to a stored obfuscated hash digest.

17. The method of claim 12, wherein the hashing is performed at a first machine and the moving is performed at a second machine.

18. The method of claim 16, wherein the hashing and moving is performed at a first machine and the comparing is performed at a second machine.

19. The method of claim 12, wherein the obfuscating instructions are unique to a machine on which the obfuscating instructions are stored.

20. The method of claim 19, wherein the obfuscating instructions are only accessible by the machine on which it is stored.
